(19) United States
(12) Patent Application Publication
(10) Pub. No.: US 2010/0263461 A1
(43) Pub. Date: Oct. 21, 2010

(54) DIE-CAST ARTICLE QUALITY JUDGING METHOD, DIE-CASTING MACHINE SELECTING METHOD, AND DIE-CASTING CONDITION DETERMINING METHOD

(75) Inventor: Masayuki Murakami, Hyogo (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: TOYO MACHINERY & METAL CO., LTD., Akashi-shi, Hyogo (JP)

(21) Appl. No.: 12/741,701

(22) PCT Filed: Oct. 23, 2008
(86) PCT No.: PCT/JP2008/069192

§ 371 Date: May 6, 2010

(30) Foreign Application Priority Data
Dec. 27, 2007 (JP) 2007-336635

(51) Int. Cl. G01N 33/20

(52) U.S. Cl. 73/865.8

(57) ABSTRACT

The present invention provides: a die-cast quality judging method that can easily judge whether or not a prescribed quality is satisfied; a die-casting machine selecting method of judging whether or not plural prescribed qualities are satisfied; and a die-casting condition determining method of determining die-casting conditions.

It is judged that inequalities to be restricted by judging standard values Q_{fLB}² and Q_{aUB}² are not true from the positional relation between a process point 13 that is an intersection of a machine characteristic line 11 and a die characteristic line 12 and straight lines 21, 22, 23, 24, and 25 and thus it is judged that the inequalities to restrict an upper limit of a filling time and a lower limit of an air vent exhaust velocity are not true. That is, it is judged that prescribed qualities are not satisfied with regard to misrun and gas inclusion.

### FIELD OF THE INVENTION

The present invention relates to: a die-cast quality judging method of judging whether or not a die-casting product cast under set die-casting conditions satisfies a prescribed quality; a die-casting machine selecting method of judging whether or not plural prescribed qualities are satisfied; and a die-casting condition determining method of determining die-casting conditions.

### BACKGROUND ART

Die-casting prevails widely as a technology for casting a product at a high speed with a high degree of accuracy. Then plural products are assorted in accordance with mold locking force by a die-casting machine maker.

When you buy a new die-casting machine or when you cast a new product with an already bought die-casting machine, you need to judge whether or not it is possible to cast a die-casting product of prescribed quality. Here, a die-cast quality is judged by nests, fins, cold folds, and cold shut for example. In general, a larger machine is more likely to satisfy a prescribed quality but the price increases. Hence it is important to select a machine that satisfies a prescribed quality and is less expensive.

Whether or not a prescribed quality is satisfied is judged through the following procedure. Firstly, the conditions of a machine, a die assembly, and molten metal are set. The conditions are collectively called die-casting conditions (casting methods). Successively, under the die-casting conditions, a value of a parameter used for quality judgment (called a “quality judging parameter”) is compared with a judging standard value functioning as a standard for judging whether or not a prescribed quality is satisfied and thereby whether or not the prescribed quality is satisfied is judged. The procedure is hereunder explained more specifically.

FIG. 11 is a table showing quality judging parameters, inequalities to restrict the quality judging parameters, and prescribed qualities satisfied when the inequalities are true. The inequalities are formed so that the quality judging parameters (the left-hand sides of the inequalities) may be restricted by the judging standard values (the right-hand sides of the inequalities) functioning as the standards for judging whether or not the prescribed qualities are satisfied. Then, when the inequalities are true, the prescribed qualities are satisfied. FIG. 12 is a table showing the expressions to obtain the judging standard values. Here, the meanings of the symbols are shown in FIG. 14. Then the symbols are known widely and thus the explanations thereof are omitted.

Explanations are made on the basis of the case where a filling time (T_{f}) is adopted as an example of the quality judging parameters. In this case, the judging standard value is T_{fUB} (the upper limit of the filling time). When the inequality (T_{f}≦Tₐₘ) to restrict the quality judging parameter (T_{f}) by the judging standard value (T_{fUB}) is true, it is judged that the prescribed quality of “few misruns” is satisfied. Here, the judging standard value T_{fUB} is obtained from the expression T_{fUB}=K_{f}×Rₘ² as shown in the table of FIG. 12. Here, K_{f} and Rₘ represent a material coefficient and a product thickness respectively (refer to FIG. 14).

The selection of a die-casting machine is carried out by judging the qualities of plural quality judging parameters as stated above and evaluating whether or not a cast die-casting product satisfies the plural prescribed qualities. When the selection of a die-casting machine is carried out and even one of the quality judging parameters not satisfying a prescribed quality exists, it is judged that it is impossible to cast a product of the prescribed qualities. On this occasion, the die-casting conditions are changed and the selection of a die-casting machine is carried out again. The change of the die-casting conditions and the selection of a die-casting machine are repeated until all the quality judging parameters satisfy all the prescribed qualities and finally die-casting conditions satisfying all the prescribed qualities are obtained.

Meanwhile, in quality judgment, it is sometimes necessary to use a value of molten metal filling pressure and a square value of a molten metal flow rate at actual casting. On this occasion, a P-Q² diagram (refer to Non-patent Documents 1 and 2) is used in many cases. The P-Q² diagram is explained hereunder.

FIG. 13 is a graph showing an example of the P-Q² diagram. In the P-Q² diagram (1), a machine characteristic line 11 and a die characteristic line 12 are drawn on a two-dimensional coordinate plane expressing a molten metal filling pressure (P) on the vertical axis 51 and a square of a molten metal flow rate (Q²) on the horizontal axis 52. The machine characteristic line 11 is a straight line showing the casting capability of a die-casting machine used and is represented by the expression P=P_{O}×(1−Q²/Q_{O}²). Here, P_{O} and Q_{O} are represented by the expressions P_{O}=P_{acc}×A_{c}/Aₛ and Q_{O}=V_{sO}×Aₛ respectively and P_{acc} represents an accumulator pressure, V_{sO} an injection velocity at blanking, A_{c} an injection cylinder cross section, and Aₛ a sleeve cross section. The die characteristic line 12 is a characteristic line intrinsic to a die assembly and is represented by the expression P=B×Q². Here, B is represented by the expression B=Dₘ/(2×A_{g}²×C_{g}²) and Dₘ represents a molten metal density, A_{g} a gate cross section, and C_{g} a flow rate coefficient.

In the P-Q² diagram (1), the intersection 13 of the machine characteristic line 11 and the die characteristic line 12 is called a “process point”. The coordinate value Q₁² of the process point 13 on the horizontal axis is the square value of a molten metal flow rate at actual casting and the coordinate value P₁ on the vertical axis is a molten metal filling pressure at the actual casting.

Non-patent Document 3 discloses a method for determining die-casting conditions by judging whether or not a process point exists within a process window in a P-Q² diagram and judging whether or not a J-Factor is in a prescribed range. It is possible to determine die-casting conditions by repeating the selection of a die-casting machine and the change of die-casting conditions until prescribed qualities are satisfied.
Non-patent Document 1: “Encyclopedia of Die-casting” supervised by 50th Anniversary Editorial Committee of Japan Diecasting Association, published by Keikinzoku Tsushin AL Co., Ltd., 2005, p. 356-357
Non-patent Document 2: “Aluminum Alloy Die-Casting—The Technology and Troubleshooting” authored by Tomonobu Sugano and Torazou Uehara, published by Kallos Publishing Co., Ltd., 1988, P. 138-143
Non-patent Document 3: “Application of PQ² Diagram and J-Factor to Evaluate Parameters for High-Pressure Die Casting Process” authored by Yoshio Kaneuchi, Hitachi Metals Technical Review, published by Hitachi Metals, Ltd., 2007, Vol. 23, p. 27-32

### DISCLOSURE OF THE INVENTION

### Problems to be Solved by the Invention

A P-Q² diagram and a graph of a J-Factor are used in the method described in Non-patent Document 3. A problem of the method using such plural graphs has been that it is necessary to refer to plural graphs in order to grasp characteristics and the method is very burdensome.

Further, by the method described in Non-patent Document 3, it is necessary to judge the change of the process point of a P-Q² diagram in a two-dimensional region (a process window) and hence a problem has been that it is necessary to confirm the values on both the vertical and horizontal axes and the method is very burdensome.

The present invention has been established in view of the above problems and an object of the present invention is to provide: a die-cast quality judging method that can easily judge whether or not a prescribed quality is satisfied; a die-casting machine selecting method of judging whether or not plural prescribed qualities are satisfied by using the die-cast quality judging method; and a die-casting condition determining method of determining die-casting conditions.

### Means for Solving the Problems

The die-cast quality judging method according to Claim 1 is a die-cast quality judging method of judging whether or not a die-casting product cast under set die-casting conditions satisfies a prescribed quality on the basis of whether or not an inequality to restrict the value of a quality judging parameter by a judging standard value functioning as a standard for judging whether or not the prescribed quality is satisfied is true; and is characterized in that whether or not the prescribed quality is satisfied is judged on the basis of whether or not an inequality modified from the inequality so as to restrict a square value of a molten metal flow rate at actual casting by a judging standard value is true.

By the die-cast quality judging method according to Claim 1, an inequality to restrict the value of a quality judging parameter by a judging standard value is modified to an inequality to restrict the square value of a molten metal flow rate at actual casting by a judging standard value and whether or not a prescribed quality is satisfied is judged on the basis of whether or not the modified inequality is true.

Explanations are made more specifically. Here, an inequality Z≧Z_{B} to restrict a value of a quality judging parameter Z by a judging standard value Z_{B} is given. When the quality judging parameter Z is represented by the expression Z=F(Q₁) by using a function F (here, F is a bijection) of a molten metal flow rate Q₁ at actual casting, the inequality to restrict the quality judging parameter becomes the expression F(Q₁)≧Z₃. The inequality is modified to an inequality (Q₁²≧Q_{B}² or Q₁²≦Q_{B}²) to restrict Q₁². Here, Q_{B}² is a judging standard value to restrict Q₁² in the modified inequality. Here, the orientation of the inequality sign in the modified inequality is determined by the function F.

Meanwhile, it is obvious that the similar modification can be applied also in the case where an inequality to restrict a value of a quality judging parameter Z by a judging standard value Z_{B} is the expression Z≦Z_{B} (the case where the inequality sign is reversed from the above case) and hence the explanations are omitted. The modified inequality is the same restricting expression as the inequality before modification and hence it is possible to judge whether or not a prescribed quality is satisfied on the basis of whether or not the modified inequality is true.

The die-cast quality judging method according to Claim 2 is characterized in that, in Claim 1 the quality judging parameter is a filling time; and, when an inequality to restrict a value of the filling time by a judging standard value functioning as a standard for judging whether or not the prescribed quality is satisfied is given, a value obtained by dividing a filling volume in the set die-casting conditions by the judging standard value to restrict the value of the filling time and squaring the divided value is used as a judging standard value to restrict the square value of a molten metal flow rate at actual casting in the modified inequality.

By the die-cast quality judging method according to Claim 2, an inequality to restrict a filling time is modified to an inequality to restrict the square value of a molten metal flow rate at actual casting by a judging standard value and whether or not a prescribed quality is satisfied is judged on the basis of whether or not the modified inequality is true.

Explanations are made more specifically. Here, an inequality T_{f}≦T_{fUB} to restrict a value of a filling time T_{f} by a judging standard value T_{fuB} is given. Since the filling time T_{f} is represented by the expression T_{f}=V_{f}/Q₁ by using a molten metal flow rate Q₁ at actual casting and a filling volume V_{f}, the above inequality becomes V_{f}/Q₁≦T_{fUB} and can be modified to Q₁²≧Q_{fLB}². Here, Q_{fLB}² is represented by the expression Q_{fLB}²=(V_{f}/T_{fUB})². The modified inequality Q₁²≧Q_{fLB}² is the same restricting expression as the inequality T_{f}≦T_{fuB} before modification and hence it is possible to judge whether or not a prescribed quality is satisfied on the basis of whether or not the modified inequality is true.

The die-cast quality judging method according to Claim 3 is characterized in that, in Claim 1 the quality judging parameter is a gate velocity; and, when an inequality to restrict a value of the gate velocity by a judging standard value functioning as a standard for judging whether or not the prescribed quality is satisfied is given, a value obtained by multiplying the judging standard value to restrict the value of the gate velocity by a gate cross section in the set die-casting conditions and squaring the multiplied value is used as a judging standard value to restrict the square value of a molten metal flow rate at actual casting in the modified inequality.

By the die-cast quality judging method according to Claim 3, an inequality to restrict a gate velocity is modified to an inequality to restrict the square value of a molten metal flow rate at actual casting by a judging standard value and whether or not a prescribed quality is satisfied is judged on the basis of whether or not the modified inequality is true.

Explanations are made more specifically. Here, an inequality V_{g}≧V_{gLB} to restrict a value of a gate velocity V_{g} by a judging standard value V_{gLB} is given. Since the gate velocity V_{g} is represented by the expression V_{g}=Q₁/A_{g} by using a molten metal flow rate Q₁ at actual casting and a gate cross section A_{g}, the above inequality becomes Q₁/A_{g}≧V_{gLB} and can be modified to Q₁²≧Q_{gLB}². Here, Q_{gLB}² is represented by the expression Q_{gLB}²=(V_{gLB}×A_{g})². The modified inequality Q₁²≧Q_{gLB}² is the same restricting expression as the inequality V_{g}≧V_{gLB} before modification and hence it is possible to judge whether or not a prescribed quality is satisfied on the basis of whether or not the modified inequality is true.

Further, when the inequality sign is reversed, it is also possible to make judgment likewise. Here, an inequality V_{g}≦V_{gUB} to restrict a value of a gate velocity V_{g} by a judging standard value V_{guB} is given. Since the gate velocity V_{g} is represented by the expression V_{g}=Q₂/A_{g} by using a molten metal flow rate Q₁ at actual casting and a gate cross section A_{g}, the above inequality becomes Q₁/A_{g}≦V_{guB} and can be modified to Q₁²≦Q_{guB}². Here, Q_{guB}² is represented by the expression Q_{guB}² (V_{gUB}×A_{g})². The modified inequality Q₁²≦Q_{gUB}² is the same restricting expression as the inequality V_{g}≦V_{gUB} before modification and hence it is possible to judge whether or not a prescribed quality is satisfied on the basis of whether or not the modified inequality is true.

The die-cast quality judging method according to Claim 4 is characterized in that, in Claim 1 the quality judging parameter is a fin forming injection velocity; and, when an inequality to restrict a value of the fin forming injection velocity by a judging standard value functioning as a standard for judging whether or not the prescribed quality is satisfied is given, a value obtained by multiplying the judging standard value to restrict the value of the injection velocity by a sleeve cross section in the set die-casting conditions and squaring the multiplied value is used as a judging standard value to restrict the square value of a molten metal flow rate at actual casting in the modified inequality.

By the die-cast quality judging method according to Claim 4, an inequality to restrict a fin forming injection velocity that is an injection velocity at which fins begin to be generated is modified to an inequality to restrict the square value of a molten metal flow rate at actual casting by a judging standard value and whether or not a prescribed quality is satisfied is judged on the basis of whether or not the modified inequality is true.

Explanations are made more specifically. Here, an inequality Vₛ≦V_{sUB} to restrict a value of a fin forming injection velocity Vₛ by a judging standard value V_{sUB} is given. Since the injection velocity Vₛ is represented by the expression Vₛ=Q₁/Aₛ by using a molten metal flow rate Q₁ at actual casting and a sleeve cross section Aₛ, the above inequality becomes Q₁/Aₛ≦V_{sUB} and can be modified to Q₁²≦Q_{sUB}². Here, Q_{sUB}² is represented by the expression Q_{sUB}²=(V_{sUB}×Aₛ)². The modified inequality Q₁²≦Q_{sUB}² is the same restricting expression as the inequality Vₛ≦V_{sUB} before modification and hence it is possible to judge whether or not a prescribed quality is satisfied on the basis of whether or not the modified inequality is true.

The die-cast quality judging method according to Claim 5 is characterized in that, in Claim 1 the quality judging parameter is an air vent exhaust velocity; and, when an inequality to restrict a value of the air vent exhaust velocity by a judging standard value functioning as a standard for judging whether or not the prescribed quality is satisfied is given, a value obtained by multiplying the judging standard value to restrict the value of the air vent exhaust velocity by an air vent cross section in the set die-casting conditions and squaring the multiplied value is used as a judging standard value to restrict the square value of a molten metal flow rate at actual casting in the modified inequality.

By the die-cast quality judging method according to Claim 5, an inequality to restrict an air vent exhaust velocity that is a velocity at which a gas is ejected from an air vent is modified to an inequality to restrict the square value of a molten metal flow rate at actual casting by a judging standard value and whether or not a prescribed quality is satisfied is judged on the basis of whether or not the modified inequality is true.

Explanations are made more specifically. Here, an inequality Vₐ≦V_{aUB} to restrict a value of an air vent exhaust velocity Vₐ by a judging standard value V_{aUB} is given. Since the air vent exhaust velocity Vₐ is represented by the expression Vₐ=Q₁/Aₐ by using a molten metal flow rate Q₁ at actual casting and an air vent cross section Aₐ, the above inequality becomes Q₁/Aₐ≦V_{aUB} and can be modified to Q₁²≦Q_{aUB}². Here, Q_{aUB}² is represented by the expression Q_{aUB}²=(V_{aUB}×Aₐ)². The modified inequality Q₁²≦Q_{auB}² is the same restricting expression as the inequality Vₐ≦V_{aUB} before modification and hence it is possible to judge whether or not a prescribed quality is satisfied on the basis of whether or not the modified inequality is true.

The die-cast quality judging method according to Claim 6 is characterized in that, in any one of Claims 1 to 5, the coordinate value on the horizontal axis of a process point that is an intersection of a machine characteristic line and a die characteristic line in a P-Q² diagram under the set die-casting conditions is set so as to be the square value of a molten metal flow rate at the actual casting.

By the die-cast quality judging method according to Claim 6, a P-Q² diagram is drawn under the set die-casting conditions and the coordinate value on the horizontal axis of a process point that is the intersection of a machine characteristic line and a die characteristic line is obtained as the square value of a molten metal flow rate at actual casting.

The die-cast quality judging method according to Claim 7 is characterized in that, in any one of Claims 1 to 5 a straight line representing a judging standard value to restrict the square value of a molten metal flow rate at actual casting in the modified inequality is drawn in a P-Q² diagram under the set die-casting conditions; two divided regions are formed in the P-Q² diagram by using the straight line representing the judging standard value as a border line; and whether or not the prescribed quality is satisfied is judged on the basis of the region in which the process point that is the intersection of a machine characteristic line and a die characteristic line in the P-Q² diagram is located.

By the die-cast quality judging method according to Claim 7, whether or not a prescribed quality is satisfied is judged by visually judging the magnitude relation between the coordinate value of a process point on the horizontal axis (namely, the square value of a molten metal flow rate at actual casting) and a judging standard value of the modified inequality in a P-Q² diagram.

Explanations are made more specifically. FIGS. 3 and 4 are graphs formed by drawing straight lines showing judging standard values in P-Q² diagrams respectively. Here, the symbols 11 and 12 are a machine characteristic line and a die characteristic line respectively and the coordinate value on the horizontal axis of a process point 13 that is the intersection is the square value Q₁² of a molten metal flow rate at actual casting. Further, the symbol 20 represents a straight line showing a judging standard value Q_{B}² in a modified inequality.

In the case where the modified inequality is represented by the expression Q₁²≧Q_{B}², when the process point 13 is in the region on the right side of the straight line 20 as shown in FIG. 3, the modified inequality is true and hence it is judged that a prescribed quality is satisfied and, when the process point 13 is in the region on the left side of the straight line 20 as shown in FIG. 4, the modified inequality is not true and hence it is judged that a prescribed quality is not satisfied. On the other hand, in the case where the modified inequality is represented by the expression Q₁²≦Q_{B}², when the process point 13 is in the region on the right side of the straight line 20 as shown in FIG. 3, the modified inequality is not true and hence it is judged that a prescribed quality is not satisfied and, when the process point 13 is in the region on the left side of the straight line 20 as shown in FIG. 4, the modified inequality is true and hence it is judged that a prescribed quality is satisfied.

A die-casting machine selecting method according to Claim 8 is characterized in that, with regard to each of plural quality judging parameters, whether or not a prescribed quality is satisfied is judged by using the die-cast quality judging method according to any one of Claims 1 to 7.

By the die-casting machine selecting method according to Claim 8, all the inequalities to restrict values of quality judging parameters are modified to inequalities to restrict the square value Q₁² of a molten metal flow rate at actual casting and whether or not prescribed qualities are satisfied is judged on the basis of whether or not the modified inequalities are true.

The die-casting machine selecting method according to Claim 9 is a die-casting machine selecting method of judging whether or not the prescribed quality is satisfied by using the die-cast quality judging method according to Claim 7 with regard to each of plural quality judging parameters; and is characterized in that straight lines representing judging standard values to restrict the square value of a molten metal flow rate at actual casting in all the modified inequalities are drawn in an identical P-Q² diagram.

By the die-casting machine selecting method according to Claim 9, all the straight lines showing judging standard values in modified inequalities are drawn in an identical P-Q² diagram and whether or not prescribed qualities are satisfied is judged respectively.

A die-casting condition determining method according to Claim 10 is characterized in that, with regard to the plural quality judging parameters, when a quality judging parameter not satisfying the prescribed quality exists as a result of judging whether or not the prescribed qualities are satisfied by using the die-casting machine selecting method according to Claim 8 or 9, the set die-casting conditions are changed.

By the die-casting condition determining method according to Claim 10, die-casting conditions are changed when a quality judging parameter not satisfying a prescribed quality exists and hence, by repeating the procedure, it is possible to obtain die-casting conditions satisfying all prescribed qualities.

### EFFECTS OF THE INVENTION

By the present invention, whether or not a prescribed quality is satisfied is judged on the basis of whether or not an inequality to restrict the square value of a molten metal flow rate at actual casting by a judging standard value, in place of an inequality to restrict a quality judging parameter by a judging standard value, is true. That is, it is possible to judge whether or not a prescribed quality is satisfied by using the square value of a molten metal flow rate that is generally used for selecting a die-casting machine.

Further, by the present invention, whether or not a prescribed quality is satisfied is judged on the basis of whether or not an inequality to restrict the square value of a molten metal flow rate at actual casting by a judging standard value, in place of an inequality to restrict a filling time by a judging standard value, is true. That is, it is possible to judge whether or not a prescribed quality stipulated by a filling time is satisfied by using the square value of a molten metal flow rate that is generally used for selecting a die-casting machine.

Further, according to the present invention, whether or not a prescribed quality is satisfied is judged on the basis of whether or not an inequality to restrict the square value of a molten metal flow rate at actual casting by a judging standard value, in place of an inequality to restrict a gate velocity by a judging standard value, is true. That is, it is possible to judge whether or not a prescribed quality stipulated by a gate velocity is satisfied by using the square value of a molten metal flow rate that is generally used for selecting and evaluating a die-casting machine.

Further, by the present invention, whether or not a prescribed quality is satisfied is judged on the basis of whether or not an inequality to restrict the square value of a molten metal flow rate at actual casting by a judging standard value, in place of an inequality to restrict a fin forming injection velocity by a judging standard value, is true. That is, it is possible to judge whether or not a prescribed quality stipulated by an injection velocity is satisfied by using the square value of a molten metal flow rate that is generally used for selecting a die-casting machine.

Further, by the present invention, whether or not a prescribed quality is satisfied is judged on the basis of whether or not an inequality to restrict the square value of a molten metal flow rate at actual casting by a judging standard value, in place of an inequality to restrict an air vent exhaust velocity by a judging standard value, is true. That is, it is possible to judge whether or not a prescribed quality stipulated by an air vent exhaust velocity is satisfied by using the square value of a molten metal flow rate that is generally used for selecting a die-casting machine.

Further, by the present invention, since a P-Q² diagram is drawn under the set die-casting conditions and the coordinate value on the horizontal axis of a process point that is the intersection of a machine characteristic line and a die characteristic line is obtained as the square value of a molten metal flow rate at actual casting, it is possible to easily obtain the square value of a molten metal flow rate at actual casting. Further, since it is possible to easily obtain the square value of a molten metal flow rate at actual casting, it is possible to easily judge whether or not an inequality to restrict the square value of a molten metal flow rate at actual casting by a judging standard value is true and whether or not a prescribed quality is satisfied.

Further, by the present invention, since whether or not a prescribed quality is satisfied is judged by visually judging the magnitude relation between the coordinate value on the horizontal axis of a process point that is the intersection of a machine characteristic line and a die characteristic line (namely, the square value of a molten metal flow rate at actual casting) and the judging standard value of a modified inequality in a P-Q² diagram, it is necessary to pay attention only to relation on the horizontal axis (namely, only to one-dimensional relation) in the P-Q² diagram and it is possible to easily make the judgment.

Further, by the present invention, since all the inequalities to restrict values of quality judging parameters are modified to inequalities to restrict the square value Q₁² of a molten metal flow rate at actual casting and whether or not prescribed qualities are satisfied is judged on the basis of whether or not the modified inequalities are true, all the prescribed qualities are judged by using the square value Q₁² of a molten metal flow rate at actual casting and thus it is possible to easily judge whether or not the prescribed qualities are satisfied. This is because, whereas it has heretofore been necessary to obtain all the values of quality judging parameters (for example, a filling time, a gate velocity, a fin forming injection velocity, an air vent exhaust velocity, and others) in addition to judging standard values in the case of judging the inequalities that are not modified and use the values of the quality judging parameters as they are, according to the present invention, it is necessary only to obtain the square value Q₁² of a molten metal flow rate at actual casting in addition to judging standard values and it is possible to reduce the number of the values to be obtained into nearly half.

Further, according to the present invention, since all the straight lines are drawn in a single P-Q² diagram, it is not necessary to refer to plural figures but necessary to refer to a single P-Q² diagram at judgment and thus it is possible to make judgment easily.

Further, by the present invention, since die-casting conditions satisfying all the prescribed qualities can be obtained by repeating the judgment on whether or not an inequality is true and the change of die-casting conditions, it is possible to determine the die-casting conditions easily.

### BRIEF DESCRIPTION OF THE DRAWINGS

FIG. 1 is a table showing quality parameters, inequalities to restrict the square value of a molten metal flow rate at actual casting corresponding to the quality parameters, and prescribed qualities satisfied when the inequalities are true in Embodiments 1 to 3 according to the present invention.

FIG. 2 is a table showing expressions to obtain judging standard values in Embodiments 1 to 3 according to the present invention.

FIG. 3 is a P-Q² diagram according to the present invention.

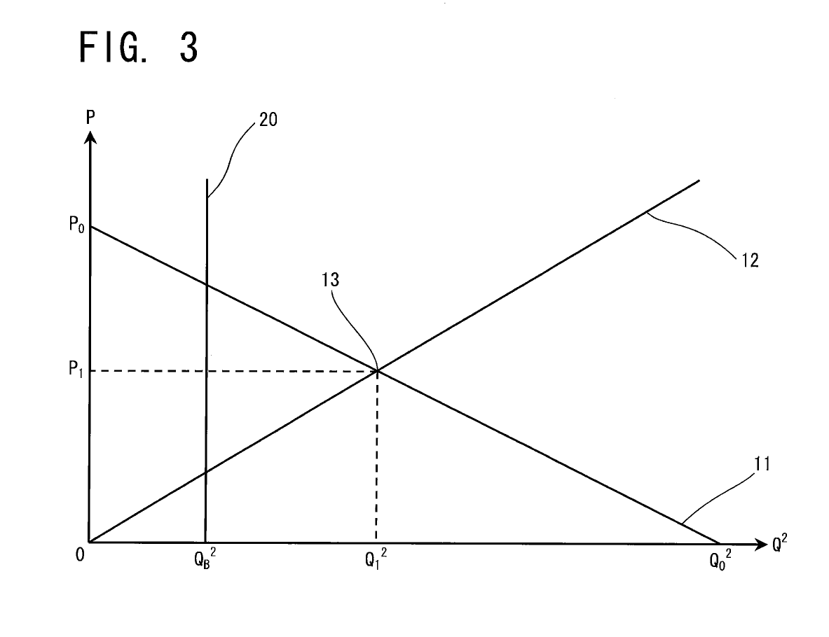

FIG. 4 is a P-Q² diagram according to the present invention.

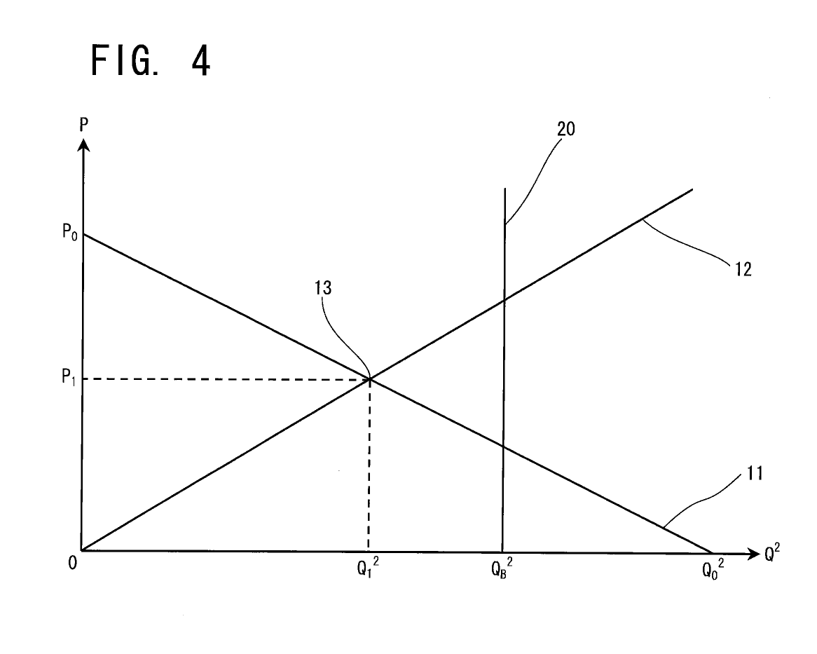

FIG. 5 is a P-Q² diagram in Embodiments 2 and 3 according to the present invention.

FIG. 6 is a P-Q² diagram in Embodiments 2 and 3 according to the present invention.

FIG. 7 is a flowchart showing the process flow of a die-cast evaluation method in Embodiment 1 according to the present invention.

FIG. 8 is a flowchart showing the process flow of a die-cast evaluation method in Embodiment 2 according to the present invention.

FIG. 9 is a flowchart showing the process flow of a die-casting condition determining method in Embodiment 3 according to the present invention.

FIG. 10 is a table showing the relation between inequalities and measures taken when the inequalities are not true in a die-casting condition determining method in Embodiment 3 according to the present invention.

FIG. 11 is a table showing quality judging parameters, inequalities to restrict the quality judging parameters, and prescribed qualities satisfied when the inequalities are true.

FIG. 12 is a table showing expressions to obtain judging standard values.

FIG. 13 is a graph showing an example of a P-Q² diagram.

FIG. 14 is a table explaining symbols.

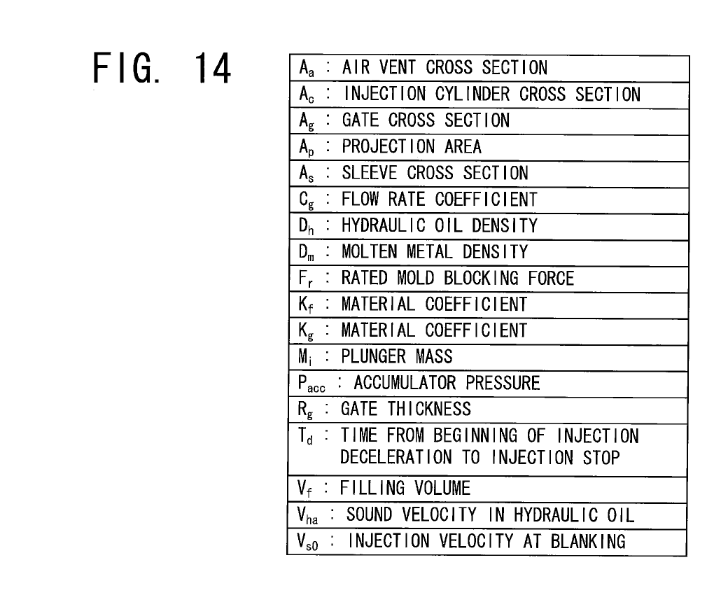

### EXPLANATION OF REFERENCE NUMERALS AND SYMBOLS

1 P-Q² diagram
11 Machine characteristic line
12 Die characteristic line
13 Process point
20 Straight line representing judging standard value Q_{B}²
21 Straight line representing judging standard value Q_{fLB}²
22 Straight line representing judging standard value Q_{gLB}²
23 Straight line representing judging standard value Q_{gUB}²
24 Straight line representing judging standard value Q_{suB}²
25 Straight line representing judging standard value Q_{aUB}²
51 Vertical axis
52 Horizontal axis

### BEST MODE FOR CARRYING OUT THE INVENTION

Embodiments according to the present invention are explained in reference to drawings. Here, the embodiments below are only concrete examples of the present invention and the present invention is not limited to the embodiments below.

### Embodiment 1

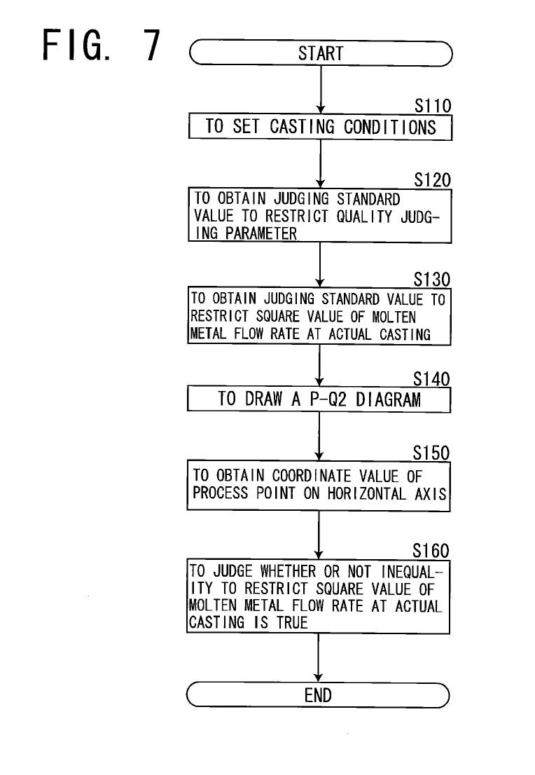

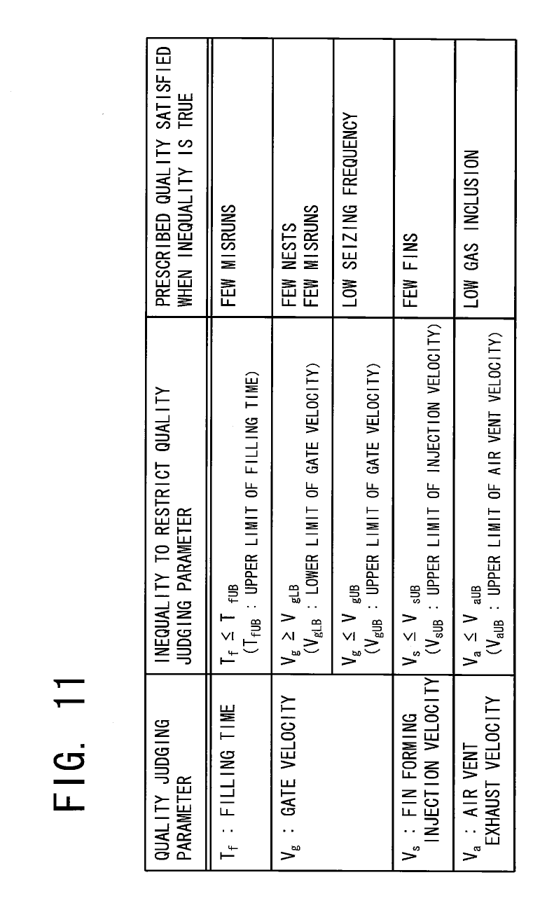

FIG. 7 is a flowchart showing the process flow of the present embodiment. Firstly at Step S110, die-casting conditions (casting methods) are set up and thereafter the processing proceeds to Step S120. FIG. 11 is a table showing die-casting conditions. Each of the values is set up as shown in the table.

At Step S120, judging standard values to restrict quality judging parameters are obtained and the processing proceeds to Step S130. FIG. 11 is a table showing quality judging parameters, inequalities to restrict the quality judging parameters, and prescribed qualities satisfied when the inequalities are true. The quality judging parameters are shown in the left column of the table and a filling time, a gate velocity, a fin forming injection velocity that is an injection velocity at which fins begin to be generated, and an air vent exhaust velocity that is a velocity at which a gas is ejected from an air vent are used in the present embodiment. The inequalities to restrict the quality judging parameters are shown in the center column and an inequality to restrict the upper limit of the filling time, an inequality to restrict the lower limit of the gate velocity, an inequality to restrict the upper limit of the gate velocity, an inequality to restrict the upper limit of the fin forming injection velocity, and an inequality to restrict the upper limit of the air vent exhaust velocity are used in the present embodiment. The right-hand sides of the inequalities represent the judging standard values respectively. The prescribed qualities satisfied when the inequalities shown in the center column are true are shown in the right column respectively.

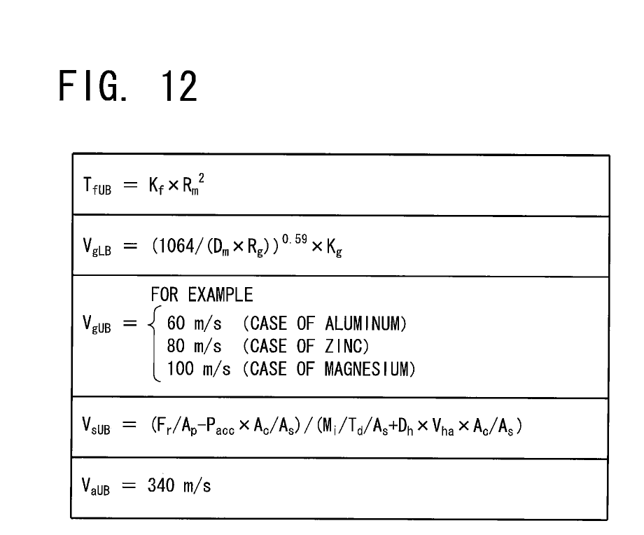

FIG. 12 is a table showing the expressions to obtain the upper limits and the lower limit (namely, judging standard values to restrict the quality judging parameters) in the center column of the table shown in FIG. 11. The judging standard values to restrict the quality judging parameters are obtained by using the expressions shown in the table.

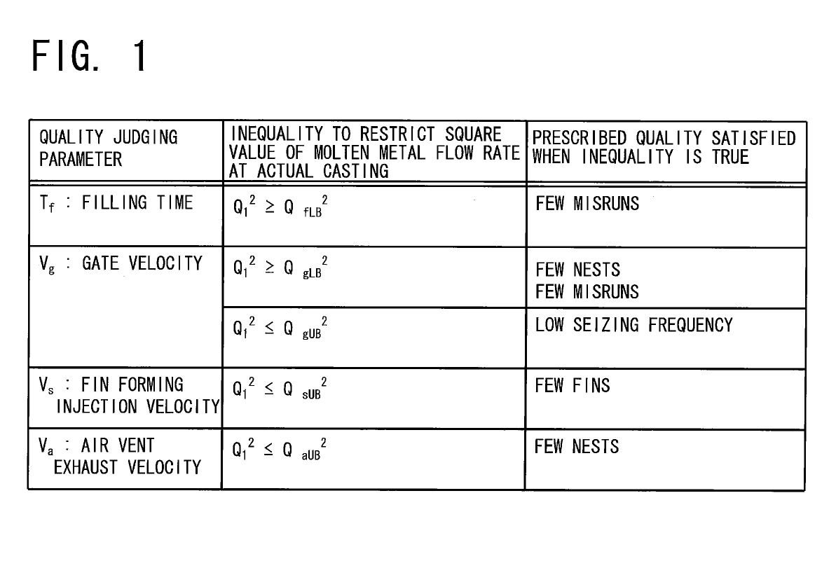

At Step S130, judging standard values to restrict the square value of a molten metal flow rate at actual casting are obtained and the processing proceeds to Step S140. FIG. 1 is a table showing the quality parameters, inequalities to restrict the square value of a molten metal flow rate at actual casting corresponding to the quality parameters, and the prescribed qualities satisfied when the inequalities are true. The quality judging parameters are shown in the left column of the table and a filling time, a gate velocity, an injection velocity, and an air vent velocity are used in the present embodiment. The inequalities modified from the inequalities to restrict the quality judging parameters (the inequalities shown in the center column in FIG. 11) so as to restrict the square value of a molten metal flow rate at actual casting are shown in the center column. Further, the right-hand sides of the inequalities represent the judging standard values to restrict the square value of a molten metal flow rate at actual casting respectively. The prescribed qualities satisfied when the inequalities in the center column are true are shown in the right column respectively.

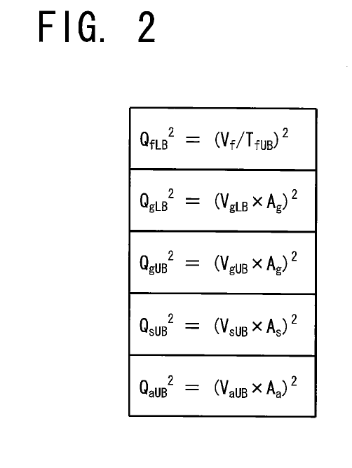

FIG. 2 is a table showing the expressions to obtain the upper limits and the lower limits (namely, the judging standard values to restrict the square value of a molten metal flow rate at actual casting) in the center column of the table shown in FIG. 1. The judging standard values to restrict the square value of a molten metal flow rate at actual casting are obtained by using the expressions shown in the table.

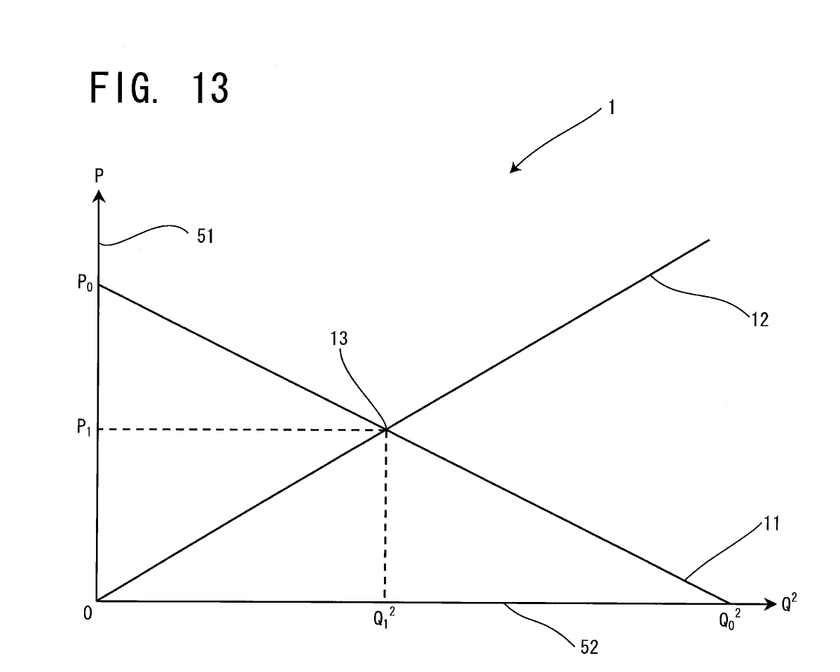

At Step S140, a P-Q² diagram (refer to FIG. 13) is drawn and the processing proceeds to Step S150. At Step S150, the coordinate value on the horizontal axis of a process point that is the intersection of a machine characteristic line and a die characteristic line in the P-Q² diagram drawn at Step S140 is obtained and the processing proceeds to Step S160. Here, the coordinate value of a process point on the horizontal axis represents the square value of a molten metal flow rate at actual casting.

At Step S160, whether or not the inequalities to restrict the square value of a molten metal flow rate at actual casting are true is judged and the processing ends. More specifically, whether or not the inequalities in the center column shown in FIG. 1 are true is judged by using the square value (Q₁²) of a molten metal flow rate at actual casting obtained at Step S150 and the judging standard values (Q_{fLB}², Q_{gLB}², Q_{guB}², Q_{sUB}², and Q_{aUB}²) obtained at Step S130.

By applying such processing, it is possible to: judge whether or not the inequalities to restrict the square value of a molten metal flow rate at actual casting are true; hence judge whether or not inequalities to restrict quality judging parameters are true; and judge whether or not prescribed qualities are satisfied.

### Embodiment 2

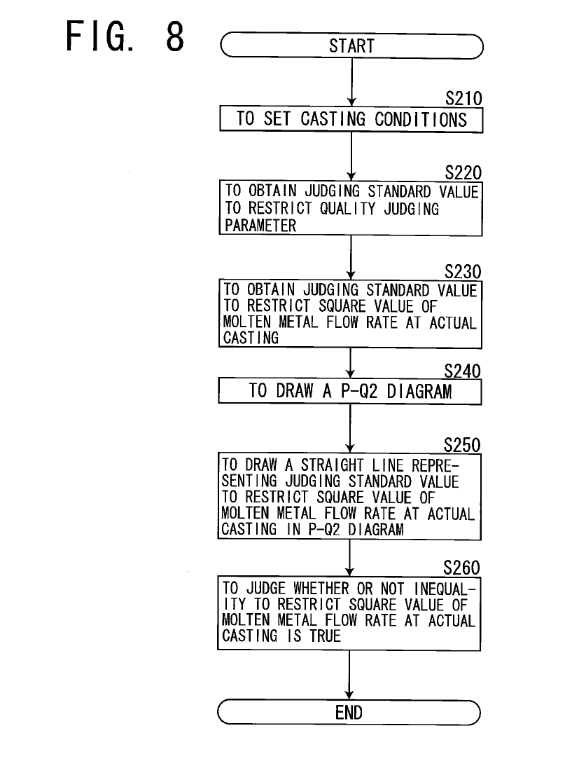

FIG. 8 is a flowchart showing the process flow of the present embodiment. The steps of Step S210 to Step S240 are identical to the steps of Step S110 to Step S140 in FIG. 7 (Embodiment 1) respectively and hence the explanations are omitted.

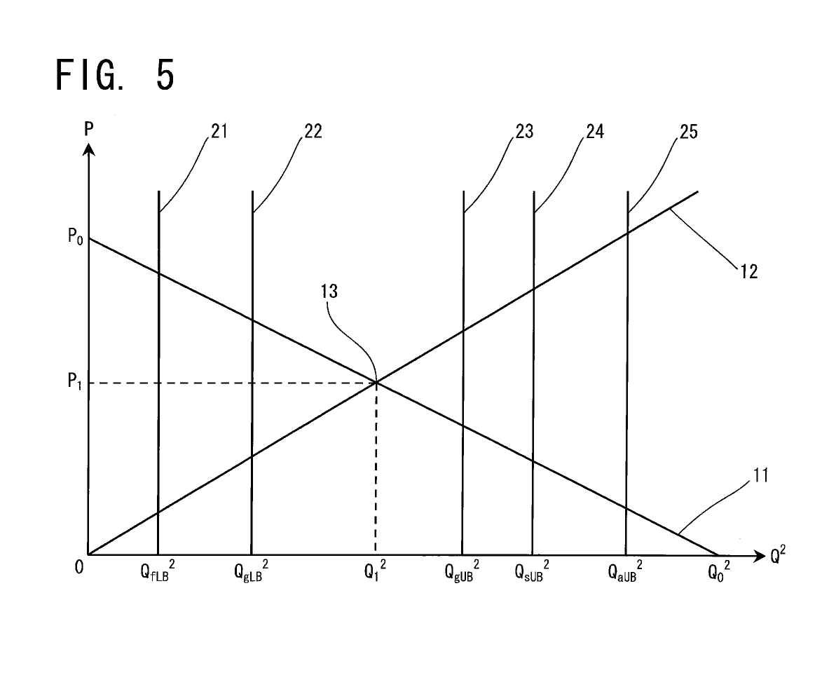

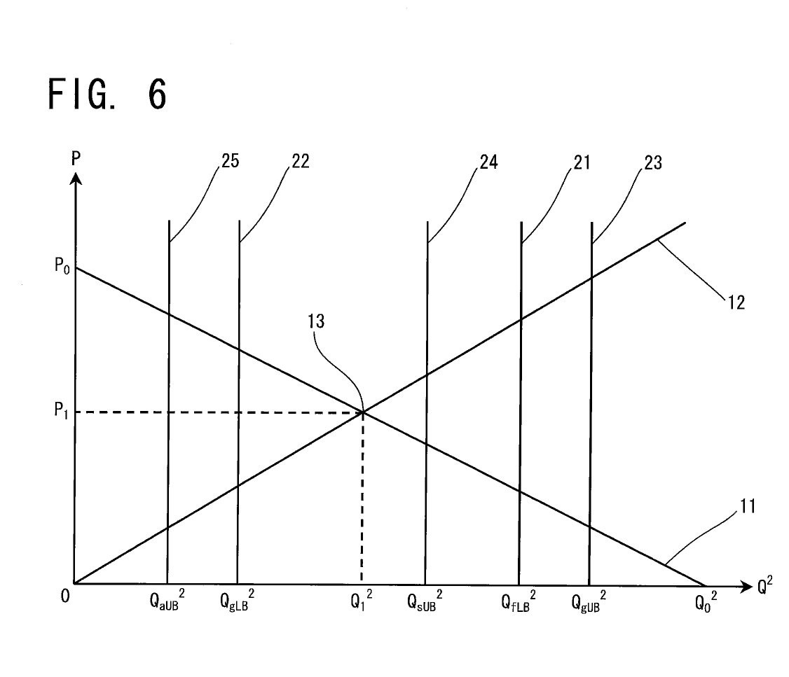

At Step S250, straight lines representing judging standard values to restrict the square value of a molten metal flow rate at actual casting are drawn in a P-Q² diagram drawn at Step S240 and the processing proceeds to Step S260. FIGS. 5 and 6 are graphs showing P-Q² diagrams in which judging standard values are drawn respectively. Here, symbols 21, 22, 23, 24, and 25 are straight lines representing the judging standard values Q_{fLB}², Q_{gLB}², Q_{gUB}², Q_{sUB}², and Q_{aUB}² in the inequalities shown in the center column of the table shown in FIG. 1 respectively. Further, the coordinate value on the horizontal axis of a process point 13 that is the intersection of a machine characteristic line 11 and a die characteristic line 12 represents the square value of a molten metal flow rate at actual casting.

At Step S260, whether or not the inequalities to restrict the square value of a molten metal flow rate at actual casting are true is judged and the processing ends. In FIG. 5, from the positional relation between the process point 13 and the straight lines 21, 22, 23, 24, and 25, it is judged that all the inequalities (the inequalities to restrict the square value of a molten metal flow rate at actual casting) shown in the center column of the table shown in FIG. 1 are true. That is, it is judged that all the prescribed qualities are satisfied.

Meanwhile, in FIG. 6, from the positional relation between the process point 13 and the straight lines 21, 22, 23, 24, and 25, it is judged that the inequalities (Q₁²≧Q_{fLB}² and Q₁²≦Q_{auB}²) restricted by the judging standard values Q_{fLB}² and Q_{aUB}² are not true and that the inequalities (T_{f}≦T_{fUB} and Vₐ≦V_{aUB}) to restrict the upper limit of a filling time and the upper limit of an air vent exhaust velocity are not true. That is, it is judged that the prescribed qualities on misrun and gas inclusion are not satisfied.

By applying such processing, it is possible to: judge whether or not the inequalities to restrict the square value of a molten metal flow rate at actual casting are true; hence judge whether or not inequalities to restrict quality judging parameters are true; and judge whether or not prescribed qualities are satisfied.

### Embodiment 3

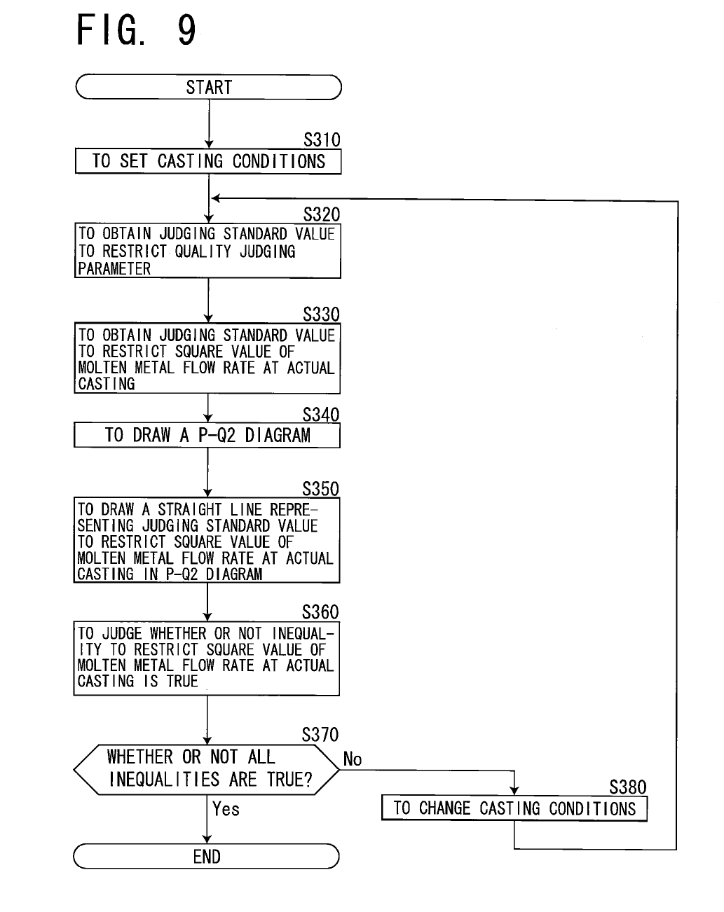

FIG. 9 is a flowchart showing the process flow of the present embodiment. The steps of Step S310 to Step S360 are identical to the steps of Step S210 to Step S260 in FIG. 8 (Embodiment 2) respectively and hence the explanations are omitted.

At Step S370, from the result of the judgment at Step S360, whether or not all the inequalities are true is judged and, when all the inequalities are true, the processing ends, and if not, the processing proceeds to Step S380.

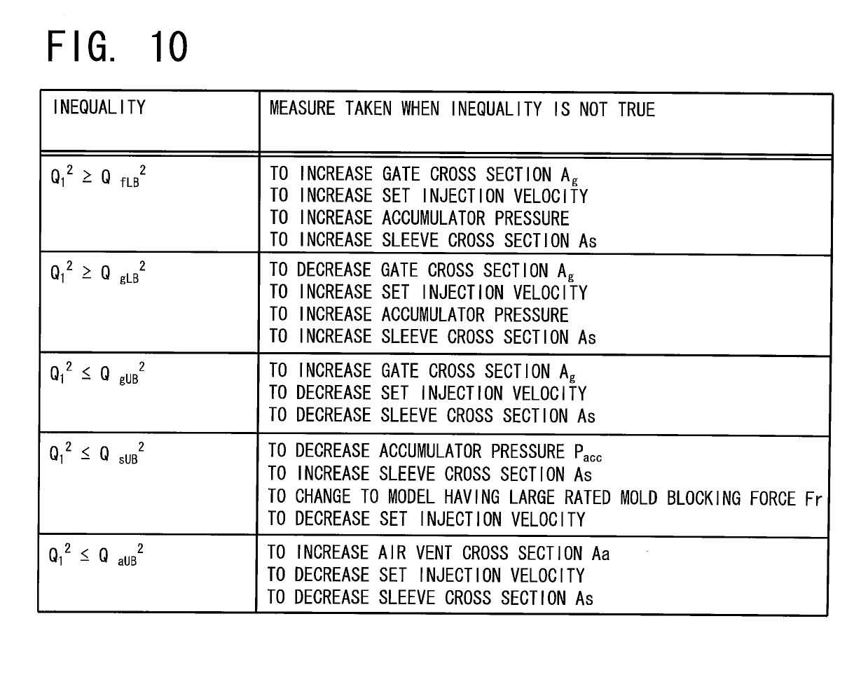

At Step S380, the die-casting conditions are changed and the processing proceeds to Step S320. FIG. 10 is a table showing the relation between inequalities and measures taken when the inequalities are not true. For example, when the inequality Q₁²≧Q_{fLB}² is not true, measures such as the increase of a gate cross section A_{g} and the like are taken.

In this way, it is possible to determine die-casting conditions by repeating the judgment of whether or not prescribed qualities are satisfied and the change of the die-casting conditions until all the inequalities become true.

Here, the method for judging a die-cast quality according to the present invention corresponds to the judgment method for quality parameters in the above Embodiments 1 to 3.

As stated above, the present invention makes it possible to provide: a die-cast quality judging method that can easily judge whether or not a prescribed quality is satisfied; a die-casting machine selecting method of judging whether or not plural prescribed qualities are satisfied by using the die-cast quality judging method; and a die-casting condition determining method of determining die-casting conditions.

Here, the processing (including judgment) in all the above embodiments either may be applied by manual operation or may be automatically computed and operated with a computer or a computer built in a die-casting machine.

Further, the straight lines representing the judging standard values in a P-Q² diagram may be drawn by changing colors. In particular, judgment becomes easier by changing colors between the case of restricting the upper limit and the case of restricting the lower limit of the square value of a molten metal flow rate at actual casting.

1. A die-cast quality judging method of judging whether or not a die-casting product cast under set die-casting conditions satisfies a prescribed quality on the basis of whether or not an inequality to restrict the value of a quality judging parameter by a judging standard value functioning as a standard for judging whether or not the prescribed quality is satisfied is true, wherein whether or not the prescribed quality is satisfied is judged on the basis of whether or not an inequality modified from the inequality so as to restrict a square value of a molten metal flow rate at actual casting by a judging standard value is true.

2. The die-cast quality judging method according to claim 1, wherein the quality judging parameter is a filling time; and, when an inequality to restrict a value of the filling time by a judging standard value functioning as a standard for judging whether or not the prescribed quality is satisfied is given, a value obtained by dividing a filling volume in the set die-casting conditions by the judging standard value to restrict the value of the filling time and squaring the divided value is used as a judging standard value to restrict the square value of a molten metal flow rate at actual casting in the modified inequality.

3. The die-cast quality judging method according to claim 1, wherein the quality judging parameter is a gate velocity; and, when an inequality to restrict a value of the gate velocity by a judging standard value functioning as a standard for judging whether or not the prescribed quality is satisfied is given, a value obtained by multiplying the judging standard value to restrict the value of the gate velocity by a gate cross section in the set die-casting conditions and squaring the multiplied value is used as a judging standard value to restrict the square value of a molten metal flow rate at actual casting in the modified inequality.

4. The die-cast quality judging method according to claim 1, wherein the quality judging parameter is a fin forming injection velocity; and, when an inequality to restrict a value of the fin forming injection velocity by a judging standard value functioning as a standard for judging whether or not the prescribed quality is satisfied is given, a value obtained by multiplying the judging standard value to restrict the value of the injection velocity by a sleeve cross section in the set die-casting conditions and squaring the multiplied value is used as a judging standard value to restrict the square value of a molten metal flow rate at actual casting in the modified inequality.

5. The die-cast quality judging method according to claim 1, wherein the quality judging parameter is an air vent exhaust velocity; and, when an inequality to restrict a value of the air vent exhaust velocity by a judging standard value functioning as a standard for judging whether or not the prescribed quality is satisfied is given, a value obtained by multiplying the judging standard value to restrict the value of the air vent exhaust velocity by an air vent cross section in the set die-casting conditions and squaring the multiplied value is used as a judging standard value to restrict the square value of a molten metal flow rate at actual casting in the modified inequality.

6. The die-cast quality judging method according to any one of claims 1 to 5, wherein the coordinate value on the horizontal axis of a process point that is an intersection of a machine characteristic line and a die characteristic line in a P-Q² diagram under the set die-casting conditions is set so as to be the square value of a molten metal flow rate at actual casting.

7. The die-cast quality judging method according to any one of claims 1 to 5, wherein a straight line representing a judging standard value to restrict the square value of a molten metal flow rate at actual casting in the modified inequality is drawn in a P-Q² diagram under the set die-casting conditions; two divided regions are formed in the P-Q² diagram by using the straight line representing the judging standard value as a border line; and whether or not the prescribed quality is satisfied is judged on the basis of the region in which the process point that is the intersection of a machine characteristic line and a die characteristic line in the P-Q² diagram is located.

8. A die-casting machine selecting method wherein, with regard to each of a plurality of quality judging parameters, whether or not the prescribed quality is satisfied is judged by using the die-cast quality judging method according to claim 1.

9. A die-casting machine selecting method of judging whether or not the prescribed quality is satisfied by using the die-cast quality judging method according to claim 1 with regard to each of a plurality of quality judging parameters, wherein straight lines representing judging standard values to restrict the square value of a molten metal flow rate at actual casting in all the modified inequalities are drawn in an identical P-Q² diagram.

10. A die-casting condition determining method wherein, with regard to the plurality of quality judging parameters, when a quality judging parameter not satisfying the prescribed quality exists as a result of judging whether or not the prescribed qualities are satisfied by using the die-casting machine selecting method according to claim 8 or 9, the set die-casting conditions are changed.
